(19) United States
(12) Patent Application Publication
(10) Pub. No.: US 2025/0031947 A1
(43) Pub. Date: Jan. 30, 2025

(54) DISTAL TIPS OF MEDICAL DEVICES AND RELATED METHODS

(71) Applicant: Boston Scientific Scimed, Inc., Maple Grove, MN (US)

(72) Inventors: Austin Grant JOHNSON, Worcester, MA (US); Allison KUMARASENA, Wellesley, MA (US)

(73) Assignee: Boston Scientific Scimed, Inc., Maple Grove, MN (US)

(21) Appl. No.: 18/780,605

(22) Filed: Jul. 23, 2024

Related U.S. Application Data
(60) Provisional application No. 63/515,940, filed on Jul. 27, 2023.

(51) Int. Cl. A61B 1/00; A61B 1/008

(52) U.S. Cl. CPC A61B 1/00096; A61B 1/008

(57) ABSTRACT

Medical devices and related methods are described, including a medical device that includes a shaft including a working channel and a distal tip. The distal tip may include an assembly that includes an imaging device and an elevator. The assembly may be movable from a lowered configuration to an angled configuration to move the imaging device and the elevator from the lowered configuration to the angled configuration.

### CROSS-REFERENCE TO RELATED APPLICATIONS

This application claims the benefit of priority of U.S. Provisional Patent Application No. 63/515,940, filed on Jul. 27, 2023, the entirety of which is incorporated herein by reference.

### TECHNICAL FIELD

Various aspects of this disclosure relate generally to distal tips of medical devices and related methods. In particular, aspects of this disclosure relate to rotating distal tips of scope devices, among other aspects.

### BACKGROUND

A scope device, such as a duodenoscope, an endoscope, or other scope device, may include a handle and a shaft, and the shaft may be insertable into a body lumen of a subject. The shaft may terminate in a distal tip, which may include features such as optical components (e.g., camera, lighting), air/fluid outlets, and working channel openings. An elevator may be disposed at the distal tip and may be actuatable in order to change an orientation of a medical device/tool passed through the working channel. For example, the elevator may be rotatable or otherwise movable, while other components at the distal tip (e.g., the optical components) may be fixed.

During a medical procedure, such as an endoscopic procedure, an operator may need to maneuver the shaft of the scope device through tortuous passages of the body to reach and position the distal tip of the shaft at a target site, e.g., a target site within the gastrointestinal tract (GI). For example, in third space endoscopy, an operator may create a tunnel between the mucosa and muscularis layers of the GI tract and maneuver the shaft through the tunnel to reach the target site. In order to view the target site, the operator may need to retroflex the shaft. A tortuous, retroflexed configuration of the shaft may make it difficult for the operator to use a medical device or tool at the target site. Therefore, a need exists for distal tips of medical devices and related methods.

### SUMMARY OF THE DISCLOSURE

Each of the aspects disclosed herein may include one or more of the features described in connection with any of the other disclosed aspects, including rotating distal tips of scope devices.

According to some aspects of the present disclosure, a medical device may include a shaft including a working channel and a distal tip. The distal tip may include an assembly including an imaging device and an elevator. The assembly may be movable from a lowered configuration to an angled configuration to move the imaging device and the elevator from the lowered configuration to the angled configuration.

According to some aspects, the assembly may be movable from the lowered configuration to the angled configuration via a hinge. In some examples, the assembly may be housed within a recessed portion of a housing of the distal tip. In some examples, the elevator and the imaging device may be independently movable from the lowered configuration to the angled configuration. In some examples, the imaging device may be on a first lateral side of the distal tip and the elevator may be on a second lateral side of the distal tip. In some examples, a bottom surface of the assembly may contact an inner surface of a housing of the distal tip in the lowered configuration. In some examples, the assembly may be coupled to a control wire. In some examples, distal movement of the control wire may move the assembly towards the inner surface of the housing and proximal movement of the control wire may move the assembly away from the inner surface of the housing. In some examples, the elevator may be disposed on a first side portion of the assembly, the imaging device may be coupled to a second side portion of the assembly, the control wire may be coupled to a medial portion of the assembly, and the medial portion may be between the first side portion and the second side portion. In some examples, in the angled configuration, a longitudinal axis of the assembly may form an angle between 0° to 90° with a longitudinal axis of the shaft. In some examples, the imaging device may face distally in the lowered configuration. In some examples, the imaging device may face at least partially radially outward in the angled configuration. In some examples, the elevator may be positioned within a groove of the assembly. In some examples, when the assembly is in the angled configuration, the imaging device and the elevator may be angled in approximately a same direction. In some examples, the assembly may further include one or more lighting sources and an irrigation opening. In some examples, the one or more lighting sources and the irrigation opening may be movable from the lowered configuration to the angled configuration.

According to some aspects of the present disclosure, a medical device may include a shaft and a distal tip. The distal tip may include a housing and a body pivotably coupled to the housing. The body may include an imaging device and an elevator. Pivoting the body relative to the housing may cause an orientation of the elevator and the imaging device to change. In some examples, the body may further include an irrigation opening and one or more lighting sources. In some examples, a control wire may be coupled to the body between the elevator and the imaging device. In some examples, movement of the control wire may be configured to pivot the body relative to the housing.

According to some aspects of the present disclosure, a medical device may include a shaft and a distal tip. The distal tip may include a pivotable body having an elevator and an imaging device. A control wire may be coupled to the pivotable body between the elevator and the imaging device. In some examples, movement of the control wire may be configured to rotate the elevator and the imaging device about a hinge.

### BRIEF DESCRIPTION OF THE DRAWINGS

The accompanying drawings, which are incorporated in and constitute a part of this specification, illustrate various aspects of this disclosure and together with the description, serve to explain the principles of the disclosure.

FIGS. 1A and 1B depict aspects of an exemplary medical device, in accordance with some aspects of the present disclosure.

FIG. 1C depicts an exemplary alternative distal tip of the medical device of FIGS. 1A and 1B, in accordance with some aspects of the present disclosure.

FIGS. 2A-2C depict a distal end portion of an exemplary medical device, in a first configuration and in a second configuration, in accordance with some aspects of the present disclosure.

### DETAILED DESCRIPTION

Conventionally, operators may utilize different types of scope devices for different medical procedures. For example, upper endoscopy or esophagogastroduodenoscopy (“EGD”) may be performed using a forward-viewing endoscope. A forward viewing endoscope may include at its distal tip an imaging device, a lighting source, air/fluid outlets, and/or a working channel opening that are forward-facing (i.e., face distally). Other procedures, such as endoscopic retrograde cholangiopancreatography (“ERCP”) may be performed using a side-viewing duodenoscope. A side-viewing duodenoscope may include at its distal tip an imaging device and/or a working channel opening that are side-facing (e.g., face radially outward relative to a central longitudinal axis of a shaft of the duodenoscope).

A side-viewing medical device, such as a duodenoscope, may include an elevator. The elevator may be operatively connected to an actuator in a handle of the duodenoscope. When an operator activates the actuator, the elevator may be raised/lowered, while the imaging device remains side-facing. As an operator inserts an accessory device (e.g., an instrument or a guidewire) into a working channel of the medical device and advances the instrument through the working channel, the elevator originally may be in a lowered configuration. After the accessory device has been extended out of a distal opening of the working channel, the elevator may be raised so as to deflect a distal tip of the accessory device.

In some medical procedures, particularly those performed in the stomach, an endoscope (e.g., a forward-viewing endoscope) may traditionally be retroflexed in order to view target anatomy (e.g., a portion of the stomach near the cardia, the fundus, or proximal portions of the body of the stomach) and use accessory devices to target the anatomy. The tortuous, retroflexed configuration of the endoscope may make it difficult to perform procedures.

The medical devices disclosed herein may include an elevator, an imaging device, a lighting source, and/or air/fluid outlets, among other components, that are rotatable or otherwise movable relative to the distal tip. For example, one or more of the components (e.g., elevator, imaging device, etc.) may rotate or otherwise move together or separately. A rotatable or movable imaging device may allow for a distal end of the medical device to be in a less tortuous, retroflexed configuration during a medical procedure. For example, an imaging device and an elevator that rotate or move together may allow an operator to view a target site and control an accessory device at the target site without needing to articulate a shaft of a medical device at large angles.

One or more of the components of the medical devices disclosed herein (e.g., imaging device and lighting source) may be movable between a forward-facing configuration and a side-facing configuration at the distal tip. Thus, the medical devices disclosed herein may allow for a user to alternate between a configuration more akin to a forward/distally-facing endoscope and a configuration more akin to a side-facing duodenoscope. One or more of the components also may adopt angles intermediate of a forward-facing configuration and a side-facing configuration.

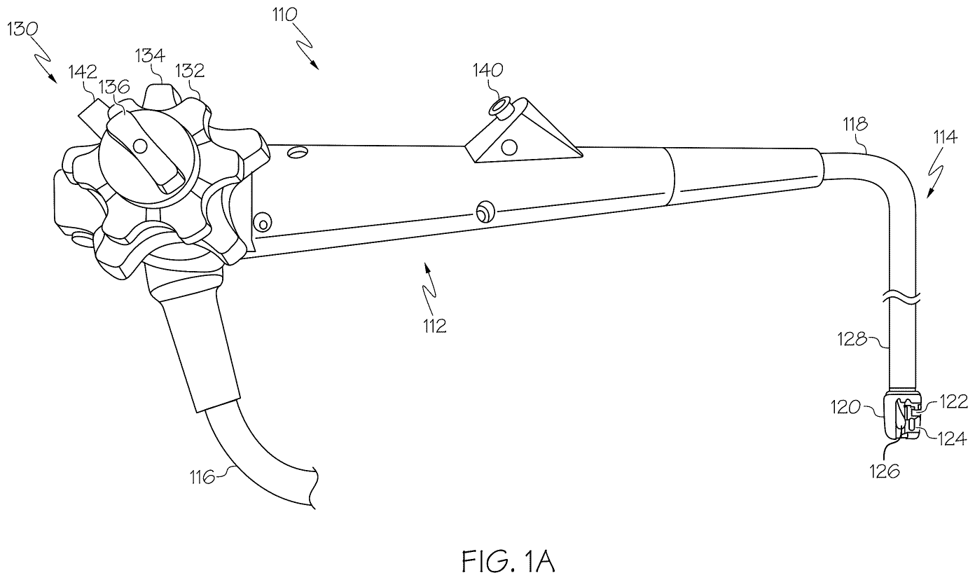

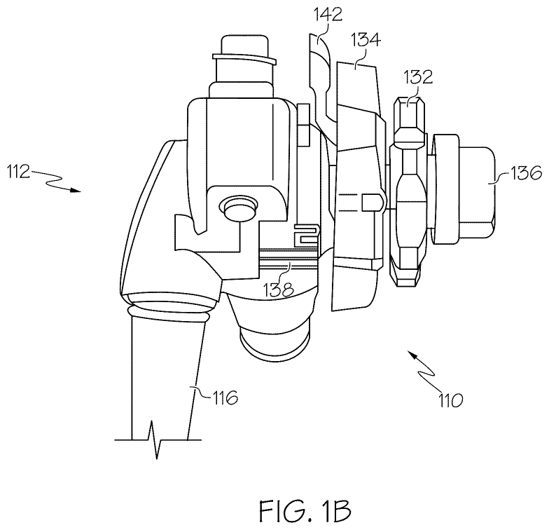

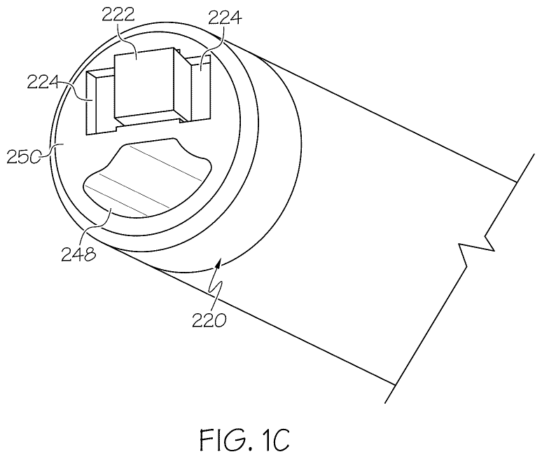

FIG. 1A depicts an exemplary medical device 110 having a handle 112 and an insertion portion 114. FIG. 1B shows a proximal end of handle 112. Medical device 110 may also include an umbilicus 116 for purposes of connecting medical device 110 to sources of, for example, air, water, suction, power, etc., as well as to image processing and/or viewing equipment. Although duodenoscopes and endoscopes (and combination devices that perform functions of duodenoscopes and endoscopes) are particularly referenced herein, the disclosure also encompasses other types of devices, such as bronchoscopes, gastroscopes, endoscopic ultrasound (“EUS”) scopes, colonoscopes, ureteroscopes, bronchoscopes, laparoscopes, cystoscopes, aspiration scopes, sheaths, catheters, or similar devices.

Insertion portion 114 may include a sheath or shaft 118 and a distal tip 120. Distal tip 120 may include an imaging device 122 (e.g., a camera) and a lighting source 124 (e.g., an LED or an optical fiber). In some examples, as shown in FIG. 1A, distal tip 120 may be side-facing (i.e., side viewing). That is, imaging device 122 and lighting source 124 may face radially outward, perpendicularly, approximately perpendicularly, or otherwise transverse to a longitudinal axis of shaft 118 and distal tip 120.

Distal tip 120 may also include an elevator 126 for changing an orientation of an accessory device or a tool inserted in a working channel of medical device 110. Elevator 126 may alternatively be referred to as a swing stand, pivot stand, raising base, or any suitable other term. Elevator 126 may be rotatable via, e.g., an actuation wire or another control element that extends from handle 112, through shaft 118, to elevator 126.

A distal portion of shaft 118 that is connected to distal tip 120 may have a steerable section 128. Steerable section 128 may be, for example, an articulation joint. Shaft 118 and steerable section 128 may include a variety of structures which are known or may become known in the art.

Handle 112 may have one or more actuators/control mechanisms 130. One or more of control mechanisms 130 may provide control over steerable section 128. One or more of control mechanisms may allow for provision of air, water, suction, etc. For example, handle 112 may include control knobs 132, 134 for left, right, up, and/or down control of steerable section 128. For example, one of knobs 132, 134 may provide left/right control of steerable section 128, and the other of knobs 132, 134 may provide up/down control of steerable section 128. Handle 112 may further include a first locking mechanism 136 and a second locking mechanism 142 (e.g., knobs or levers) for preventing steering and/or braking of steerable section 28 in at least one of an up, down, left, or right direction. Handle 112 may include a control lever 138 (see FIG. 1B). Control lever 138 may raise and/or lower elevator 126, via connection between lever 138 and an actuating wire that extends from lever 138, through shaft 118, to elevator 126. A port 140 may allow passage of a tool through port 40, into a working channel of the medical device 110, through shaft 118, to distal tip 120.

In use, an operator may insert at least a portion of shaft 118 into a body lumen of a subject. Distal tip 120 may be navigated to a procedure site in the body lumen. The operator may insert an accessory device (not shown) into port 140, and pass the accessory device through shaft 118 via a working channel to distal tip 120. The accessory device may exit the working channel at distal tip 120. The user may use control lever 138 to raise elevator 126 and angle the accessory device toward a desired location (e.g., a papilla of the pancreatico-biliary tract). The user may use the accessory device to perform a medical procedure.

FIG. 1C depicts another exemplary distal tip 220 that may be used with medical device 110 of FIGS. 1A and 1B. Distal tip 220 may be used in place of distal tip 120. Distal tip 220 may include a distalmost face 250. Distalmost face 250 may define a working channel opening 248. The working channel may extend between port 140 and working channel opening 248, such that instruments or other devices may be passed through port 140, through the working channel, and out of working channel opening 248. An instrument extending distally of working channel opening 248 may be used to perform a medical procedure on a subject.

Distal tip 220 may also include imaging components, such as one or more lighting element 224 and one or more imaging devices (e.g., camera) 222. Although two lighting elements 224 and one imaging device 222 are depicted in FIG. 1C, it will be appreciated that alternative numbers of lighting elements 224 and imaging device 222 may be utilized. Alternatively, lighting elements 224 and imaging device 222 may be combined into a single device. Lighting elements 224 may include LEDs or any suitable alternative light source. Imaging device 222 may be configured to take video and/or still images. Imaging device 222 may provide a signal to a monitor (not shown), so that an operator may view a visual image provided by imaging device 222 while navigating medical device 110 through a body lumen of a subject.

As depicted in FIG. 1C and described above, medical device 110 may be forward-facing (i.e., forward viewing). In other words, features of distal tip 220 (e.g., working channel opening 248, lighting elements 224, and imaging device 222) may face distally (i.e., forward of distalmost face 250). Medical device 110 may additionally include some components that are forward-facing and other components that are side-facing.

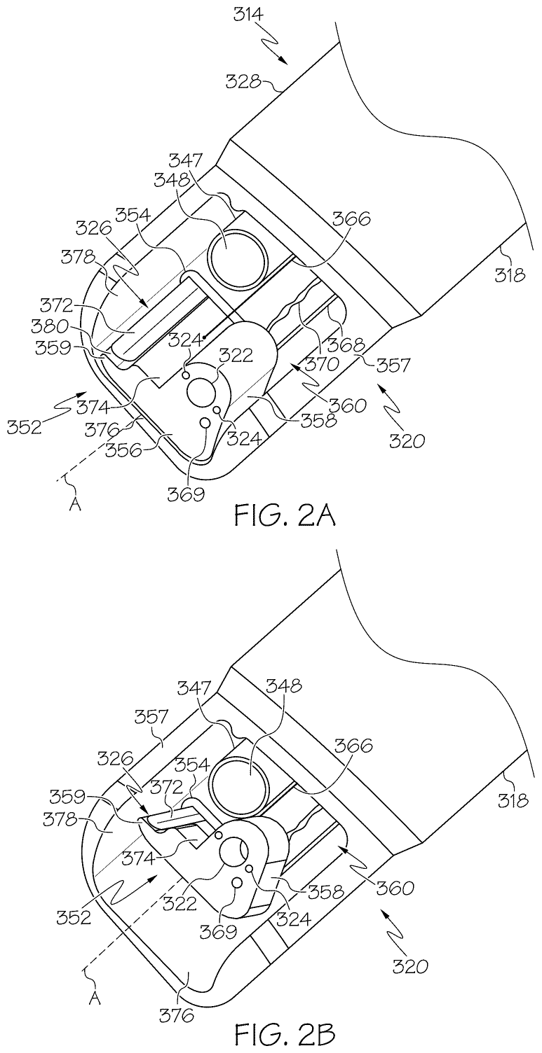

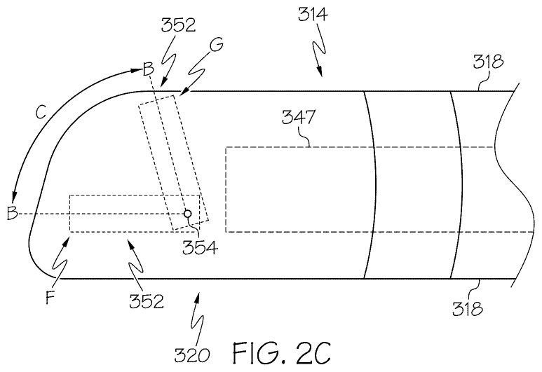

FIGS. 2A-2C depict an exemplary distal end portion of an insertion portion 314 of a medical device according to aspects of the present disclosure. Insertion portion 314 may be used in place of insertion portion 114 in combination with handle 112 of medical device 110. Insertion portion 314 may have any of the features of insertion portion 114, unless otherwise specified herein. Insertion portion 314 may include a shaft 318 (having any of the properties of shaft 118, unless stated herein) and a distal tip 320 (having any of the properties of distal tip 120, unless stated herein). Similar to shaft 118, a distal portion of shaft 318 may have a steerable section 328, which may be controlled by one or more of control mechanisms 130 of handle 112.

Distal tip 320 may include a housing 357. Housing 357 may include a recessed portion 360 defined by a bottom inner surface 376 of housing 357 and side inner surfaces 378 of housing 357. As used herein, the terms “top” and “bottom” refer to directions of FIGS. 2A and 2B. Inner surfaces 378 may be on either lateral side of distal tip 320. The term “lateral” refers to a direction that is substantially perpendicular to a longitudinal axis of shaft 318. Although only one inner surface 378 is labeled in FIGS. 2A and 2B, it will be appreciated that housing 357 may include two inner surfaces 378 on opposite sides of distal tip 320. Housing 357 may be open distal and top sides thereof, such that recessed portion 360 forms a channel having open distal and top sides. In cross-section, housing 357 may have an approximately “C” or “U” shape.

Pivotable assembly 352 may be housed within recessed portion 360 of housing 357 and coupled to housing 357 via a hinge 354. Pivotable assembly 352 may rotate or pivot about hinge 354 within recessed portion 360 from a lowered configuration, shown in FIG. 2A, to an angled configuration, shown in FIG. 2B. In some examples, hinge 354 may bias pivotable assembly 352 to the lowered configuration (FIG. 2A), in which a bottom surface of pivotable assembly 352 contacts bottom inner surface 376 of housing 357. In other aspects, hinge 354 may not be biased, and pivotable assembly 352 may remain in the angled configuration (FIG. 2B) if not otherwise actuated.

A pull wire 366 (a control wire) may extend proximally from a medial portion 374 of pivotable assembly 352, through shaft 318, and to an actuator, e.g. a lever, switch, button, or other actuator of handle 112, such as control lever 138. Medial portion 374 of pivotable assembly 352 may be positioned between a first side portion 358 and a second side portion 359 of pivotable assembly 352, each of which will be described in greater detail below. Proximal movement of pull wire 366 rotates pivotable assembly 352 about hinge 354 away from bottom inner surface 376 of housing 357 and distal movement of pull wire 366 rotates pivotable assembly 352 towards bottom inner surface 376 of housing 357. Although pull wire 366 is shown coupled to medial portion 374 of pivotable assembly 352, it will be appreciated that pull wire 366 may be coupled to any portion (e.g., side portions 358, 359) of pivotable assembly 352 to rotate pivotable assembly 352 about hinge 354.

First side portion 358 of pivotable assembly 352 may include an imaging device 322 and/or one or more lighting sources 324 disposed on a distal surface 356 of pivotable assembly 352. Although FIGS. 2A and 2B depict two lighting sources 324 and one imaging device 322, it will be appreciated that any number of lighting sources 324 and imaging devices 322 may be utilized. Imaging device 322 and/or one or more lighting sources 324 may be coupled to electrical wires 370 extending from first side portion 358, through shaft 318, and to a handle and/or an umbilicus of the medical device (e.g., handle 112 and/or umbilicus 116). In some examples, electrical wires 370 may extend through an opening (not shown) on a proximal surface of first side portion 358 and into a lumen (not shown) of first side portion 358 to connect with imaging device 322 and/or lighting source(s) 324.

First side portion 358 may also include a nozzle or opening 369 disposed on distal surface 356 to apply, e.g., gas, fluid, and/or suction at a target site. Opening 369 may be fluidly coupled to an irrigation tube 368. Irrigation tube 368 may extend through shaft 318 to a handle and/or umbilicus of the medical device (e.g., handle 112 and/or umbilicus 116). In some examples, first side portion 358 may include a lumen (not shown) therethrough, having an opening (not shown) on a proximal surface of first side portion 358. Irrigation tube 368 may extend through the lumen or be fluidly coupled to the lumen.

Second side portion 359 of pivotable assembly 352 may include an elevator 326 disposed within a groove 380. In some examples, elevator 326 may be integrally formed from a single piece of material with second side portion 359 of pivotable assembly 352. In alternatives, elevator 326 may be formed as a separate piece and be coupled to second side portion 359. Elevator 326 may include a guide surface 372 configured to receive an accessory device or a tool exiting a working channel 347. Working channel 347 may extend from distal tip 320, through shaft 318, to a handle (e.g., handle 112). Working channel 347 may terminate distally at a forward-facing opening 348 proximal to a proximal end of pivotable assembly 352. Alternatively, working channel 347 may extend distally past a proximal end of pivotable assembly 352. Working channel 347 may be positioned in alignment with elevator 326, such that an accessory device or tool may contact and move along guide surface 372 of elevator 326 as the accessory device or tool exits opening 348 of working channel 347.

Relative positions of first side portion 358, second side portion 359, and medial portion 374 shown in FIGS. 2A and 2B are merely exemplary, and portions 358, 359, and 374 may have any suitable arrangement. For example, as shown, first side portion 358 and second side portion 359 may be on opposite sides of medial portion 374 (and opposite sides of distal tip 320). In another example, side portions 358, 359 may be adjacent to one another. Side portions 358, 359 and medial portion 374 may form a single, movable body. In some examples, two or more of portions 358, 359, and 374 may be integrally formed with one another, from a same piece of material (e.g., a single molded piece). In alternatives, portions 358, 359, and 374 may be formed from separate pieces that are coupled (e.g., fixedly coupled) to one another.

Although working channel 347, portions of electrical wires 370, and portions of irrigation tube 368 are shown exposed within recessed portion 360 in FIGS. 2A and 2B, it will be appreciated that working channel 347, electrical wires 370, and/or irrigation tube 368 may be sealed to protect components of the medical device from, e.g., fluid at a target site.

In the lowered configuration (FIG. 2A), imaging device 322, one or more lighting sources 324, and/or opening 369 may face distally. In other words, they may be forward-facing (i.e., approximately parallel to or coaxial with a longitudinal axis A of shaft 318). A longitudinal axis of elevator 126 may be approximately parallel to longitudinal axis A of shaft 318.

In the angled configuration (FIG. 2B), pivotable assembly 352 may be rotated about hinge 354 by any suitable angle. In the angled configuration, imaging device 322, one or more lighting sources 324, and opening 369 may face at least partially radially outward. In other words, pivoting pivotable assembly 352 may cause an orientation of imaging device 322 and one or more lighting sources 324 to change. These components may face a direction that is transverse (i.e., not parallel to) to longitudinal axis A of shaft 318. In some examples, these components may face approximately 90 degrees from longitudinal axis A of shaft 318. In other examples, these components may face partially proximally or partially distally, at an angle between 0° and 90° relative to longitudinal axis A of shaft 318. Similarly, a longitudinal axis of elevator 126 may be transverse to longitudinal axis A of shaft 318, having any of the angles described above for imaging device 322, one or more lighting sources 324, and opening 369. Pivotable assembly 352 may be adjustable through various angles, between the lowered configuration and a maximum pivoted configuration. In examples, pivotable assembly 352 may be locked at various different angles.

In use, an operator may insert a portion of shaft 318 into a body lumen of a subject. Distal tip 320 may be navigated to a target site in the body lumen. During navigation, pivotable assembly 352 may be in the lowered configuration of FIG. 2A.

In order to view and/or treat the target site, an operator may engage an actuator, e.g., a lever, switch, button, or other actuator, such as control lever 138 of handle 112, in order to move pull wire 366 in a proximal direction to rotate pivotable assembly 352 about hinge 354 and away from bottom inner surface 376 to the angled configuration (FIG. 2B). Pull wire 366 may be moved proximally or distally to rotate pivotable assembly 352 about hinge 354 to adjust pivotable assembly 352 until pivotable assembly 352 is at a desired angle. For example, pivotable assembly 352 may be angled to allow the operator to view the target via imaging device 322, to illuminate the target site via one or more lighting sources 324, to apply fluid, gas, and/or suction to the target site via opening 369, and/or to angle an accessory device or a tool towards the target site via elevator 326. The operator may lock the position of the pivotable assembly 352 in place via a locking mechanism on the handle of the medical device.

FIG. 2C shows a schematic view of pivotable assembly 352 of distal tip 320. As shown in FIG. 2C, pivotable assembly 352 may allow for an operator to view a target site and/or position an accessory device or a tool at a target site at a range of exit angles along an arc C. For example, a longitudinal axis B of pivotable assembly 352 may form an angle with longitudinal axis A of shaft 318 and/or bottom inner surface 376 of housing 357 between approximately 0 degrees to approximately 90 degrees. As shown in FIG. 2C, in a first position F of pivotable assembly 352 (which may correspond to the lowered configuration of FIG. 2A), longitudinal axis B of pivotable assembly 352 may be approximately parallel to longitudinal axis A of shaft 318. In a second position G, pivotable assembly 352 may have an angle along arc C. In examples, pivotable assembly 352 may have a maximum angle (not specifically shown) of approximately 90 degrees along arc C. Because housing 357 may be open distal and top sides thereof, such that recessed portion 360 forms a channel having open distal and top sides, imaging device 322 may view a target site and/or a tool or accessory device may access a target site along the entire length of arc C unimpeded.

Although imaging device 322, one or more lighting sources 324, opening 369, and elevator 326 are shown as jointly rotatable or otherwise movable via pull wire 366 coupled to medial portion 374, it will be appreciated that pivotable assembly 352 may not include medial portion 374, and side portions 358, 359 may each be coupled to a respective pull wire 366 to separately rotate each side portion 358, 359 about a hinge (e.g., hinge 354). For example, first side portion 358, including imaging device 322, one or more lighting sources 324, and opening 369, may be coupled to a first pull wire 366 to rotate first side portion 358 about hinge 354 independently of second side portion 359. Second side portion 359, including elevator 326, may be coupled to another, second pull wire 366 to rotate portion 359 about hinge 354 independently of first side portion 358. Separately movable portions of pivotable assembly 352 may allow for an operator to extend a tool or an accessory device through working channel 347 and onto guide surface 372 of elevator 326 in a straight configuration, while imaging device 322, one or more lighting sources 324, and opening 369 of irrigation tube 368 may be angled. Alternatively, imaging device 322, one or more lighting sources 324, and opening 369 of irrigation tube 368 may be approximately parallel to longitudinal axis A of shaft 318, while elevator 326 may have a non-zero angle with respect to longitudinal axis A of shaft 318. In this example, the operator may lock the position of each side portion 358, 359 of the pivotable assembly 352 in place separately via a locking mechanism on a handle of the medical device (e.g., similar to handle 112). Handle 112 may include two control levers 138 for separately controlling first side portion 358 and second side portion 359.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device comprising:
a shaft comprising a working channel; and
a distal tip, wherein the distal tip includes an assembly comprising an imaging device and an elevator, and wherein the assembly is movable from a lowered configuration to an angled configuration to move the imaging device and the elevator from the lowered configuration to the angled configuration.

2. The medical device of claim 1, wherein the assembly is movable from the lowered configuration to the angled configuration via a hinge.

3. The medical device of claim 1, wherein the assembly is housed within a recessed portion of a housing of the distal tip.

4. The medical device of claim 1, wherein the elevator and the imaging device are independently movable from the lowered configuration to the angled configuration.

5. The medical device of claim 1, wherein the imaging device is on a first lateral side of the distal tip, and wherein the elevator is on a second lateral side of the distal tip.

6. The medical device of claim 1, wherein a bottom surface of the assembly contacts an inner surface of a housing of the distal tip in the lowered configuration.

7. The medical device of claim 6, wherein the assembly is coupled to a control wire, wherein distal movement of the control wire moves the assembly towards the inner surface of the housing, and wherein proximal movement of the control wire moves the assembly away from the inner surface of the housing.

8. The medical device of claim 7, wherein the elevator is disposed on a first side portion of the assembly, wherein the imaging device is coupled to a second side portion of the assembly, wherein the control wire is coupled to a medial portion of the assembly, and wherein the medial portion is between the first side portion and the second side portion.

9. The medical device of claim 1, wherein, in the angled configuration, a longitudinal axis of the assembly forms an angle between 0° to 90° relative a longitudinal axis of the shaft.

10. The medical device of claim 1, wherein the imaging device faces distally in the lowered configuration.

11. The medical device of claim 1, wherein the imaging device faces at least partially radially outward in the angled configuration.

12. The medical device of claim 1, wherein the elevator is positioned within a groove of the assembly.

13. The medical device of claim 1, wherein when the assembly is in the angled configuration, the imaging device and the elevator are angled in approximately a same direction.

14. The medical device of claim 1, wherein the assembly further comprises one or more lighting sources and an irrigation opening.

15. The medical device of claim 14, wherein the one or more lighting sources and the irrigation opening are movable from the lowered configuration to the angled configuration.

16. A medical device, comprising:
a shaft; and
a distal tip, wherein the distal tip includes:
a housing; and
a body pivotably coupled to the housing, wherein the body includes an imaging device and an elevator, and wherein pivoting the body relative to the housing causes an orientation of the elevator and the imaging device to change.

17. The medical device of claim 16, wherein the body further includes an irrigation opening and one or more lighting sources.

18. The medical device of claim 16, wherein a control wire is coupled to the body between the elevator and the imaging device, wherein movement of the control wire is configured to pivot the body relative to the housing.

19. A medical device, comprising:
a shaft; and
a distal tip, wherein the distal tip includes a pivotable body having an elevator and an imaging device, wherein a control wire is coupled to the pivotable body between the elevator and the imaging device.

20. The medical device of claim 19, wherein movement of the control wire is configured to rotate the elevator and the imaging device about a hinge.
